(19) United States
(12) Patent Application Publication
(10) Pub. No.: US 2014/0186302 A1
(43) Pub. Date: Jul. 3, 2014

(54) Nutritional Formulations and Method for Treating Diseases

(71) Applicant: Barbara L. DUNNING, Toronto (CA)

(72) Inventor: Barbara L. DUNNING, Toronto (CA)

(21) Appl. No.: 13/730,217

(22) Filed: Dec. 28, 2012

(51) Int. Cl. A61K 31/07; A61K 35/74

(52) U.S. Cl. CPC A61K 31/07; A61K 35/745; A61K 35/747
USPC 424/93.4; 514/725

(57) ABSTRACT

The present invention provides methods for treating retinal disorders using a nutraceutical composition comprising lutein and zeaxanthin. Methods for treating vitreomacular traction disorder and related disorders are disclosed.

### CROSS REFERENCE TO PRIOR APPLICATIONS

Not applicable.

### FIELD OF THE INVENTION

The field of invention relates to methods and compositions useful for treating retinal disorders in a subject including vitreomacular traction and related disorders.

### BACKGROUND OF THE INVENTION

Vitreomacular traction (VMT) is an idiopathic disorder, wherein the vitreous gel strongly adheres to the retina. With ageing, it is common for the vitreous to separate from the retina. However, if separation is incomplete, the retina is pulled by the vitreous. This “traction” force generated by vitreal pulling on the retina can cause edema within the retina, vessel damage, bleeding, optic nerve damage, retinal distortion and decreased visual acuity (VA). Adhesion size and strength of the traction force determine the resulting ocular pathology and symptoms. Continuous VMT may induce retinal lesions, leading to VMT syndrome. Complications related to VMT syndrome include, but are not limited to, epiretinal membrane (ERM), macular pucker, partial lamellar holes, macular holes, visual impairment and blindness.

Some VMT disorders are asymptomatic. Treatment for asymptomatic VMT involves regular monitoring of the patient for further progression of VMT. In cases where VMT causes reduced or distorted vision in a patient, the current standard of care is pars plana vitrectomy. Vitrectomy requires surgical removal of the vitreous from the eye and membrane peeling to relieve epiretinal traction. Complications of vitrectomy can include retinal tearing or detachment, endopthalmitis and postoperative cataracts and fibrovascular membranes.

On Oct. 17, 2012 the United States Food and Drug Administration (FDA) approved a biological agent for treatment of VMT called Jetrea™ (ocriplasmin; ThromboGenics). Ocriplasmin, a proteolytic enzyme that cleaves fibronectin, laminin and collagen, is to be provided to a patient by intravitreal injection to resolve vitreomacular adhesion, which can result in VMT. Ocriplasmin does not reverse fibrosis.

Less invasive methods of treating symptomatic VMT are desirable.

Nutrition is one feature of ocular health that has been studied in age-related ocular diseases such as age-related macular degeneration (AMD). Macular degeneration is a chronic eye disease that causes vision loss in the central field of vision. Dry macular degeneration is marked by deterioration of the deep layers of the retina. Wet macular degeneration is characterized by blood vessels that grow under the retina, leaking blood and fluid. The pathology of AMD is believed to be caused, at least in part, by oxidative damage (Beatty et al., Surv. Opthamol. 2000, 45:115-134; Cai et al., Prog. Retin. Eye Res. 2000, 29:263-271, incorporated herein by reference as if set forth in their entirety). The healthy eye contains antioxidant molecules, including enzymes, vitamins C and E, omega-3 fatty acid docosahexanic acid (DHA) and macular pigments lutein and zeaxanthin. Deficiency of antioxidants in the ageing eye is believed to be a risk factor for development of AMD (Ocular Nutrition: It's Role in Maintaining Eye Health, Module 1: Nutrition and Health of the Aging Eye, 2011, 6 pages, incorporated herein by reference as if set forth in its entirety). It follows that nutrient supplements, including antioxidants such as, zinc, vitamin C, vitamin E, beta carotene, lutein, zeaxanthin and omega-3 fatty acids, are sometimes recommended to prevent AMD progression and improve vision.

Lutein and zeaxanthin are xanthophyll carotenoid pigments found in the macula. Subjects having AMD are known to have decreased amounts of lutein and zeaxanthin in their macula. Some studies suggest that visual acuity, contrast sensitivity, and the amount of macular pigment in the human eye can be improved as a result of lutein and zeaxanthin supplementation or a combination of these xanthophylls with other antioxidants (Stiles et al. (2004) Optometry, 75:216-230, incorporated herein by reference as if set forth in its entirety). Other studies suggest that macular pigment optical density (MPOD), a measure of the amounts of lutein and zeaxanthin in the macula of the living human eye, is a marker of the health of the human eye (U.S. Patent Application Publication No. 2012/0070422, incorporated herein by reference as if set forth in its entirety).

Nutritional supplements including lutein and zeaxanthin have also been suggested to promote ocular health and treat “ocular diseases” (see, for example, U.S. Patent Application Publication Nos. 2010/0068298 and 2012/0258168, each incorporated herein by reference as if set forth in their entirety). However, the range of “ocular diseases” appears to be limited to AMD and related ocular disorders thought to be associated with oxidative stress (U.S. Patent Application Publication No. 2010/0068298). Similarly, “ocular health” is thought to decrease naturally with age, and can be compromised by oxidative stress, illness and visual stresses, such as prolonged exposure to visual display monitors (U.S. Patent Application Publication No 2012/0258168). Vitreomacular traction has not been considered in such studies of treating “ocular disease” or “ocular health”.

Nutritional treatment of VMT has not been suggested in the literature or established in the field, but is desirable as a non-invasive alternative to treatment with vitrectomy or intravitreal injections.

### SUMMARY OF THE INVENTION

In one aspect, the present invention provides a method for treating a retinal disorder comprising administering to a subject in need thereof a therapeutically effective amount of a composition comprising lutein and zeaxanthin.

In some embodiments of the aspect the retinal disorder is vitreomacular traction disorder. In some embodiments of the aspect the retinal disorder is a nutritional deficiency disorder.

In some embodiments of the aspect the therapeutically effective amount of lutein is about 10-20 mg daily. In some embodiments of the aspect the therapeutically effective amount of zeaxanthin is about 0.5-5 mg daily. In some embodiments of the aspect 20 mg lutein and 5 mg zeaxanthin are administered to the subject daily.

In some embodiments, the composition is administered orally to the subject. In some embodiments, the composition is administered daily for two to 24 months.

In some embodiments, the composition further comprises at least one of omega 3 fatty acids and probiotics. In some embodiments fatty acids are at least one of Docosahexaenoic acid (DHA) and Eicosapentaenoic acid (EPA). In some embodiments the probiotics are live microorganisms belonging to the order Lactobacilliales or the genus Bifidobacterium.

### BRIEF DESCRIPTION OF THE DRAWINGS

The features of the invention will become more apparent in the following detailed description in which reference is made to the appended drawings wherein:

FIGS. 1A-H depict ocular photos obtained from the subject of case 1. A: OD, October, 2009; B: OS, October, 2009; C: OD, November, 2010; D: OS, November, 2010; E: OD, November, 2011; F: OS, November, 2011; G: OD, May, 2012; and H: OS, May, 2012.

FIG. 2 depicts changes in retinal lesion size in the subject of case 1. Changes in retinal lesion size are indicated on the Y axis, treatment and time are indicated on the x axis. L(20)Z(0) on the Y axis refers to administration of 20 mg lutein every other day. All other treatments were administered on a daily basis.

FIGS. 3A-D depict ocular photos obtained from the subject of case. A: OD, Oct. 12, 2012; B: OS, Oct. 12, 2012; C: OD, Nov. 15, 2012; D: OS, Nov. 15, 2012.

FIGS. 4A-F depict ocular photos obtained from the subject of case 19. A: OD, September 2009; B: OS, September 2009; C: OD, September 2010; D: OS, September 2010; E: OD, April 2012; F: OS, April 2012.

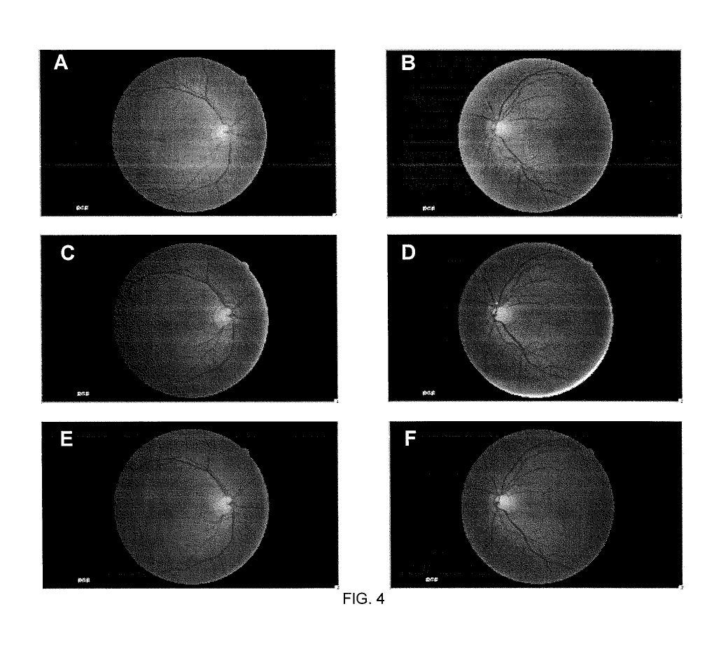

FIGS. 5 A-D depict ocular photos obtained from the subject of case 38. A: OD, January 2012; B: OS, January 2012; C: OD, July 2012; D: OS, July 2012.

FIGS. 6 A-D depict ocular photos obtained from the subject of case 41. A: OD, October, 2012; B: OS, October, 2012; C: OD, November, 2012; and D: OS, November, 2012.

### DETAILED DESCRIPTION OF THE INVENTION

The present invention is based upon the inventor's observation that vitreomacular traction (VMT) can be treated in subjects, in need thereof, by daily administration of a composition comprising lutein and zeaxanthin. A portion of the inventor's observations were presented by the inventor at a meeting of The American Academy of Optometry, Ocular Nutrition Special Interest Group, on Wednesday, Oct. 24, 2012, and by another who is not an inventor of the present invention at the same meeting on the same date.

The definitions of certain terms as used in this specification are provided below. Unless defined otherwise, all technical and scientific terms used herein generally have the same meaning as commonly understood by one of ordinary skill in the art to which this invention belongs.

As used herein, the term “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent to a subject includes any route of introducing or delivering to a subject a compound to perform its intended function. Administration can be carried out by any suitable route, including orally, intraocularly, intranasally, or topically. Administration includes self-administration and the administration by another.

As used herein the term “probiotic” refers to live microorganisms that may confer a health benefit on their host. Probiotics can be consumed as part of fermented foods or as dietary supplements. Examples of probiotic organisms include some members of the Order Lactobacillales, such as Lactobacillus spp. And members of the genus Bifidobacterium.

As used herein, the term “nutraceutical” refers to specific chemical compounds found in foods that can prevent disease or ameliorate an undesirable condition.

As used herein, the term “retinal disorder” or “disorder of the retina” refers to any impairment of normal physiological function of the retina.

As used herein, the term “nutritional deficiency disorder” refers to an impairment of normal physiological function of the retina, wherein the cause of impairment is the lack of one or more nutrients.

As used herein, the term “Oculus Dexter” or “OD” refers to the right eye of a subject.

As used herein, the term “Oculus Sinister” or “OD” refers to the left eye of a subject.

As used herein, the term “omega 3 fatty acids” refers to fats commonly found in marine and plant oils, such as fish oils, algal oil, squid oil, echium oil and flaxseed oil. Examples of omega 3 fatty acids useful in the present invention include, but are not limited to, eicosapentaenoic acid (EPA) and docosahexaenoic acid (DHA).

As used herein, the term “therapeutically effective amount” refers to a quantity sufficient to achieve a desired therapeutic and/or prophylactic effect, e.g., an amount which results in the prevention of, or a decrease in, the symptoms associated with a retinal disorder. For example, a “therapeutically effective amount” of the composition of the present invention refers to levels of the composition that, when administered to the subject on a daily basis, ameliorate, in part or in full, at least one symptom of the disorder, for example, the size of a retinal lesion.

As used herein, the term “lesion” refers to a localized change in an organ or tissue of the body. “Retinal lesions”, referred to herein, can be characterized by at least one of puckering, fibrosis, lamellar splitting, and/or dragging, swelling, bulging of retinal tissues and retinal holes.

As used herein, the terms “treating” or “treatment” or “alleviation” refers to both therapeutic treatment and prophylactic or preventative measures, wherein the object is to reverse, prevent or slow down (lessen) the targeted pathologic condition or disorder. A subject is successfully “treated” for a retinal disorder if, after receiving an effective therapeutic amount of the composition according to the methods described herein, the subject shows measurable reduction in at least one symptom or sign of a retinal disorder. It is also to be appreciated that the various modes of treatment or prevention of medical conditions as described are intended to mean “substantial”, which includes total but also less than total treatment or prevention, and wherein some biologically or medically relevant result is achieved.

As used herein, the terms “vitreomacular traction,” “vitreomacular traction disorder” and “vitreoretinal traction” refer to a conditions wherein the vitreous gel of the human eye adheres to the retina, causing pulling, or “traction”, forces on the retina that can cause ocular damage.

As used herein, the terms “units of diameter” and units made in reference to lesion size or diameter refer to units of diameter of the posterior pole of an eye, which measures ten units in diameter between the vascular arcades. A lesion can be larger than 19 units.

The terms “comprise”, “comprises”, “comprised” or “comprising” may be used in the present description. As used herein (including the specification and/or the claims), these terms are to be interpreted as specifying the presence of the stated features, integers, steps or components, but not as precluding the presence of one or more other feature, integer, step, component or a group thereof as would be apparent to persons having ordinary skill in the relevant art.

Non-invasive methods for treating vitreomacular traction (VMT) and related disorders have been highly desired. The present invention provides methods for treating VMT using a nutraceutical composition. A person of skill in the art understands that nutraceutical compounds are found in foods and can be used to prevent disease or ameliorate an undesirable condition.

In some embodiments of the present invention, the methods involve identifying a subject in need of VMT treatment and administering to the subject a therapeutically effective daily dose of a composition comprising lutein and zeaxanthin.

Subjects in need of VMT treatment typically have a retinal lesion in one or both eyes. Retinal lesions can be detected using methods known to those of skill in the art, at least for example, retinal photography, optical coherence tomography, OCT, visual acuity, Amsler grid or fundus examination

In one embodiment of the present invention, therapeutically effective dosages of lutein and zeaxanthin for treatment of VMT include about 10-20 mg lutein and about 0.5-5 mg zeaxanthin. Preferably, a ratio of 4:1 lutein: zeaxanthin is provided to a subject for treatment of VMT. An especially preferred therapeutically effective daily dose of a composition comprising lutein and zeaxanthin comprises 20 mg lutein and 5 mg zeaxanthin.

In some embodiments of the present invention, the composition is administered to the subject orally. The compositions of the invention can be formulated with suitable carriers such as starch, sucrose or lactose in tablets, capsules, solutions, powders, syrups and emulsions, or oils. Suitable optional carriers include but are not limited to, for example, fatty acids, esters and salts thereof, that can be derived from any source, such as for example, natural or synthetic oils, fats, waxes or combinations thereof. In preferred embodiments of the present invention, the source of the fatty acids is DHA or EPA, which may be provided in combination with the composition or separately.

In some embodiments of the present invention, the composition is administered daily for two to 24 months to the subject. In preferred embodiments, the composition is administered daily to the subject for 3 months. In particularly preferred embodiments of the present invention, the composition is administered to the subject daily until retinal lesions are ameliorated completely.

In one embodiment of the present invention, the method is used for treating VMT. In other embodiments of the present invention, the method is used for treating retinal disorders related to VMT, such as, for example, epiretinal membrane (ERM), macular pucker, partial lamellar holes and/or macular holes.

It is contemplated herein, that idiopathic VMT and related retinal disorders are associated with an underlying nutritional deficiency disorder, wherein the subject having the disorder is deficient in at least lutein and zeaxanthin. It is further contemplated that such nutritional deficiency can be corrected in a subject in need of VMT treatment by administering to the subject therapeutically effective amounts of lutein and zeaxanthin, daily.

It is contemplated herein that treatment of VMT with lutein and zeaxanthin can be further improved by administering to the subject one or more omega 3 fatty acid and/or probiotic.

### EXAMPLES

The present invention is further illustrated by the following examples, which should not be construed as limiting in any way.

### Example 1

### Materials and Methods

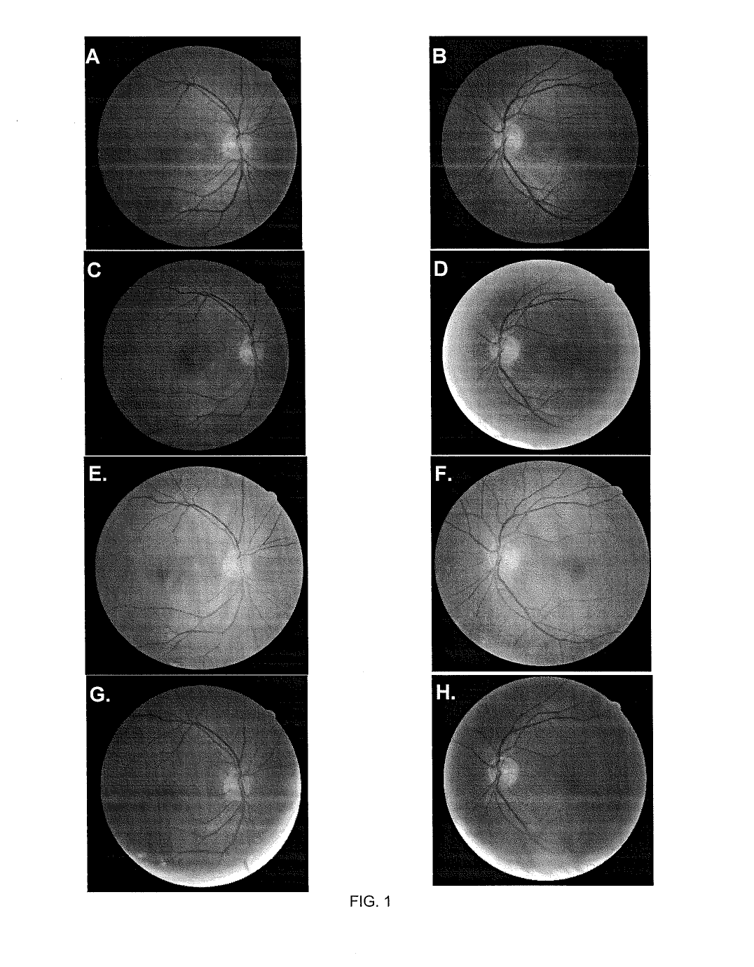

Diagnosis of vitreomacular traction (VMT): VMT can be symptomatic or asymptomatic. Complaints of reduced or distorted vision may or may not be reported by the subject. Measurement of visual acuity (VA) may or may not be normal or reduced in one or both eyes. Measurement for distortion on an Amsler grid may or may not show distortion in one or both eyes. Retinal photography may show signs of VMT in the original color composite image (e.g., upper left photo in four panel photo of FIG. 1A). Further analysis of the choroid (e.g., upper right photo in four panel photo of FIG. 1A), the retina (e.g., lower left photo in four panel photo of FIG. 1A), and the retinal nerve fibre layer (RNFL) (e.g., lower right photo in four panel photo of FIG. 1A), may reveal puckering, fibrosis, lamellar splitting, partial lamellar holes, pseudo macular holes, bulging and/or edema in the choroid, retinal and/or RNFL images. Further analysis with “3-D” images of the choroid, retina and RNFL layers may show elevations diagnostic of VMT, such as, for example, the presence, severity and/or size of puckering, partial lamellar holes, macular holes, bulging or edema. Further, “3-D” images can be useful for determining which layers of the eye tissue are affected by VMT.

Ocular imaging: retinal photographs were taken using a Canon CR-1 Digital Retinal Camera and analyzed using EyeScape Digital Imaging Software, version 7.5.5.

The settings used for taking retinal photographs were such that optimal contrast and brightness for each subject was achieved. Most retinal photographs were taken with a dilated pupil. Some subjects have naturally very large pupils. In these cases, pupil dilation may not have been required to obtain sufficient images. The Canon CR-1 has a setting for small pupils that was used as required.

### Example 2

### Case 1

Subject Description:

The subject is a male born in 1938. Subject underwent laser therapy for retinal detachment in his left eye in 2002. An ophthalmological examination in December 2008 revealed ERM in the subject's OS.

Treatment:

Subject began administration of 20 mg lutein every second day in June 2009 and continued for 4 months. From October 2009 through October 2010 the subject was administered 3 times daily, 1000 mg omega-3 fatty acids from fish oil. From November 2010 through November 2011 the subject was administered daily with 10 mg lutein and 2.0 mg zeaxanthin. From December 2011 through October 2012, the subject was administered daily with 20 mg lutein and 5 mg zeaxanthin.

Results:

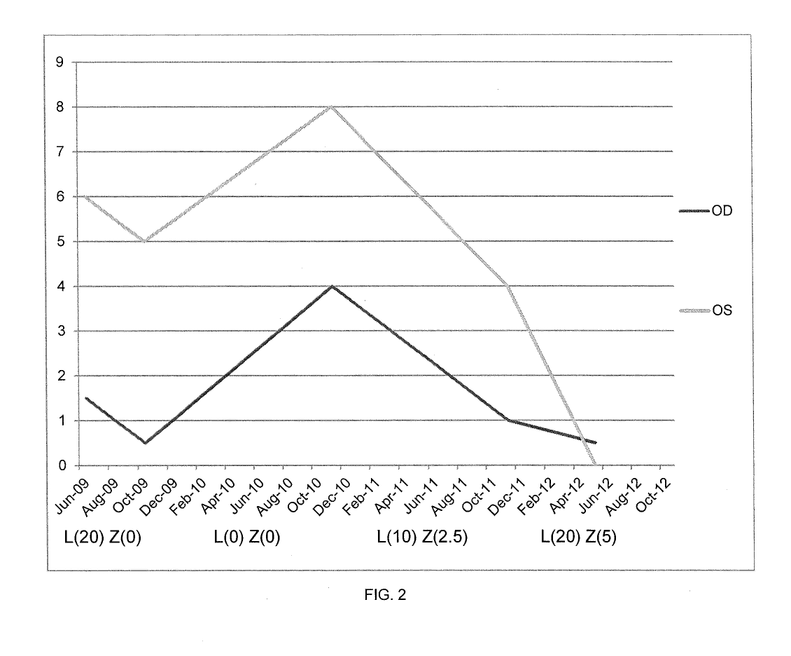

Four months of treatment with lutein (10 mg) every other day resulted in reduction of ERM OD from 1.5 units to 0.5 units and a reduction of OS macular pucker from 6.5 units to 5.0 units (FIG. 1, A, B). During the following 12 months, in the absence of supplemental lutein and zeaxanthin, lesions in the subject's right and left eyes increased (FIG. 2). The subject's ERM OD increased from 0.5 units to 4.5 units and OS macular pucker increased from 5 units to 8 units (FIGS. 1C and 1D relative to 1A and 1B, respectively). From November 2010 through November 2011, treatment with 10 mg lutein and 2.0 mg zeaxanthin, resulted in a decrease in retinal lesion size in both of the subject's eyes (FIG. 2). The subject's ERM OD decreased from 4.5 units to 2 units and OS macular pucker decreased from 8 units to 4 units (FIGS. 1E and 1F relative to FIGS. 1C and 1D, respectively). Further treatment with an increased dose of lutein (20 mg/day) and zeaxanthin (5 mg/day) for 11 months resulted in further decrease in retinal lesion size (FIGS. 1G and 1H relative to FIGS. 1E and 1F, respectively). Treatment with 20 mg/day lutein and 5 mg/day zeaxanthin was sufficient to completely ameliorate the lesion in the subject's left eye.

### Example 3

### Case 16

Subject Description:

The subject is a female born in 1949. Subject had normal retinal health in December, 2010. April 2012, the subject had ERM OS of 13.5. Oct. 12, 2012, subject had lesions in both eyes, OD 11.5 units, OS 14.5 units.

Treatment:

The subject was administered 20 mg/day lutein and 5 mg/day zeaxanthin on an irregular basis from April 2012 until Oct. 11, 2012. The subject was administered daily 20 mg lutein and 5 mg zeaxanthin from Oct. 12, 2012 through Nov. 15, 2012.

Results:

Following about one month of regular administration of lutein and zeaxanthin, the subject's lesion size and severity decreased in both eyes: OD lesion decreased from 11.5 to 10 units, the OS lesion decreased from 14.5 to 9.5 units.

### Example 4

### Case 19

Subject Description:

The subject is a male born in 1941. Subject had OD lesion of 0.5 units in September 2009. January 2010, the subject had OD and OS lesions of 0.5 units. Further, the subject has been diagnosed with diabetes in January 2010 and was being treated with metformin. September 2010, subject had no OD lesion and an OS lesion of 7.5 units. October 2011, subject's OS lesion had increased to 8.5 units. By December 2011, the subject was no longer diabetic and was no longer taking metformin. April 2012, subjects' OS lesion had decreased to 0.5 units. October 2012, subject's OS lesion had been ameliorated

Treatment:

The subject was administered 10 mg lutein and 2.0 mg zeaxanthin daily from October 2010 until October 2011. The subject was administered 20 mg lutein and 5 mg zeaxanthin daily from November 2011 through October, 2012.

Results:

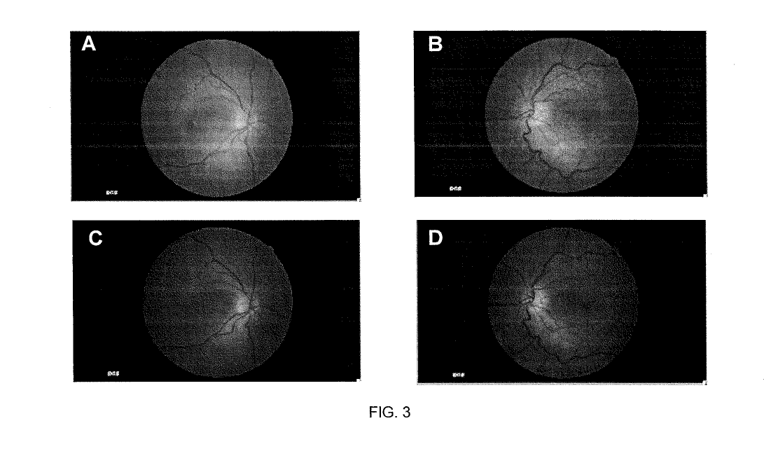

Twelve months of treatment with lutein (10 mg) and zeaxanthin (2.0 mg) resulted in an increase in OS lesion size from 7.5 units to 8.5 units (FIG. 3, C, D). Further treatment with an increased dose of lutein (20 mg/day) and zeaxanthin (5 mg/day) for 6 months was sufficient to completely ameliorate the OS lesion (FIG. 3F).

### Example 5

### Case 38

Subject Description:

The subject is a female born in 1946. The subject is diabetic. Subject had ERM OS lesion of 4.3 units in January 2010. OS lesion had increased to 8 units by January 2012. July 2012, subject's ERM OS lesion had been ameliorated.

Treatment:

The subject was administered 20 mg lutein and 5 mg zeaxanthin daily from January 2012 through July 2012.

Results:

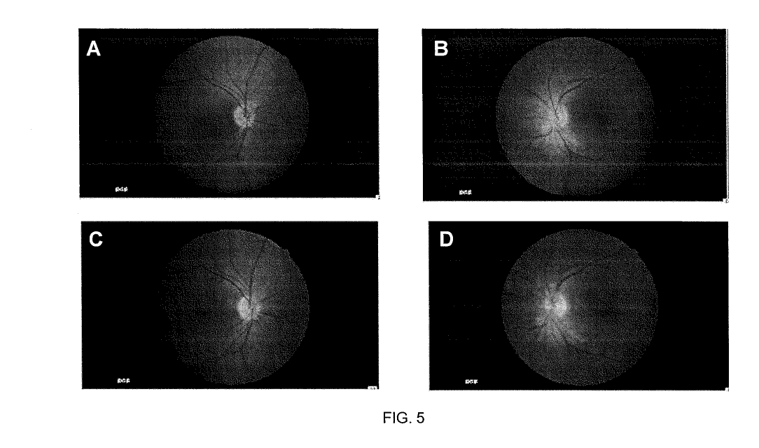

Six months of treatment with lutein (20 mg/day) and zeaxanthin (5 mg/day) was sufficient to completely ameliorate the ERM OS lesion in the subject (FIG. 5D).

### Example 6

### Case 41

Subject Description:

The subject is a male born in 1970. Subject developed central serous retinopathy in both eyes, Sep. 17, 2012. OS became symptomatic with VA 20/40 and large area of “bulging lines” on Amsler grid. Subject's OD lesion was 4 units and OS lesion was 6 units on Oct. 18, 2012. Subjects OD lesion was 4 units, but improving on Nov. 30, 2012 and subject's OS lesion had been ameliorated by the same date; OS VA 20/20, Amsler grid greatly improved relative to Oct. 18, 2012 measurement.

Treatment:

The subject was administered 10 mg lutein and 2.0 mg zeaxanthin daily for one week commencing on Oct. 11, 2012. At this time the subject was also administered daily with 360 mg EPA and 240 mg DHA. The subject was administered no lutein or zeaxanthin from October 18 through Oct. 24, 2012, at which time the subject was administered daily probiotics containing 5 billion live microorganism cells. From Oct. 25, 2012 through Nov. 30, 2012 the subject was administered daily with 20 mg lutein, 5 mg zeaxanthin, 360 mg EPA, 240 mg DHA and probiotics containing 5 billion live microorganism cells.

Results:

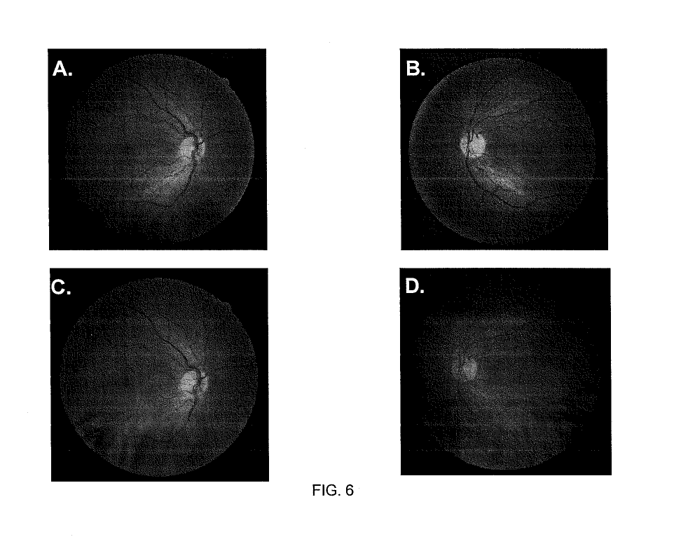

Within 6 weeks following treatment with lutein (20 mg/day) zeaxanthin (5 mg/day), probiotics and omega 3 fatty acids the subject's OS VA returned to 20/20 and the subject's Amsler distortion was greatly diminished. Further, treatment was sufficient to ameliorate the subject's OS lesion (FIG. 6D relative to FIG. 6B).

### Example 7

### Lutein and Zeaxanthin Dosage Response Trials Indicate Positive Results for Treatment of VMT, Including Retinal Lesions

Subject Description:

Data were collected from 40 subjects having symptomatic VMT over a period of 4.5 years.

Treatment:

Subjects were treated daily with lutein and zeaxanthin as described in table 1.

TABLE 1
Raw data collected from subjects during preliminary lutein zeaxanthin trial.
Plh indicates partial lamellar hole. Pucker (macular) is described based on a qualitative
score of mild to severe, wherein a score of 1 of 3 is a mild pucker, a score of 2 of 3
is a moderate pucker and a score of 3 of 3 is a sever pucker.

| | | Date of | Date of Ocular | | Zeaxanthin | OD lesion | OS lesion |
|---|---|---|---|---|---|---|---|
| Case # | Gender | Birth | Examination | Lutein (mg) | (mg) | (units) | (units) |
| 1 | M | 1939 | Jul-08 | 0 | 0 | 1.5 | 6 |
| | | | Oct-09 | 10 | 0 | 0.5 | 5 |
| | | | Nov-10 | 0 | 0 | 4.5 | 8 |
| | | | Nov-11 | 10 | 2 | 2 | 4 |
| | | | May-12 | 20 | 5 | .5 | 0 |
| | | | Oct-12 | 20 | 5 | 0.5 | 0 |
| 2 | F | 1959 | Apr-09 | 0 | 0 | 0 | 8.5 |
| | | | Nov-10 | 20 | 0 | 0 | 3 |
| | | | Mar-12 | 20 | 5 | 0 | 0 |
| | | | Sep-12 | 0 | 0 | 1 | 0.5 |
| 3 | M | 1959 | Apr-09 | 0 | 0 | 9 | 0 |
| | | | May-10 | 0 | 0 | 9 | 0 |
| | | | Jun-11 | 2.5 | 0.5 | 11.5 | 0 |
| | | | Dec-11 | 0 | 0 | 10.5 | 0 |
| | | | Jul-12 | 0 | 0 | 14 | 0 |
| 4 | F | 1951 | Aug-10 | 0 | 0 | 5 | 0 |
| | | | Jul-12 | 0 | 0 | 5 | 0 |
| 5 | F | 1941 | Jul-10 | 0 | 0 | 4.5 | 11.5 |
| | | | Jan-11 | 5 | 1 | 4 | 12 |
| | | | Aug-11 | 20 | 0.8 | 7 | 12 |
| | | | Aug-12 | 20 | 0.8 | 6.5 | 11.5 |
| 6 | M | 1950 | Feb-11 | 0 | 0 | 7 | 7 |
| | | | Aug-11 | 20 | 5 | 6.5 | 7 |
| | | | Jul-12 | 20 | 5 | 4 | 7 |
| 7 | F | 1958 | May-11 | 0 | 0 | 5 | 0 |
| | | | Nov-11 | 0 | 0 | 8 | 0 |
| | | | May-12 | 0 | 0 | 8.5 | 0 |
| 9 | M | 1928 | Jan-11 | 10 | 0.5 | 11.5 | 0.5 |
| | | | Jul-11 | 10 | 0.5 | 7.5 | 0.5 |
| | | | Jan-12 | 30 | 1.3 | 8.5 | 0.5 |
| | | | Aug-12 | 25 | 1.05 | 7.5 | 0.5 |
| 10 | F | 1949 | Jan-10 | 0 | 0 | 5.5 | 1.5 |
| | | | Jul-10 | 0 | 0 | 8.5 | 3.5 |
| | | | Jan-11 | 5 | 1 | 8.5 | 5.5 |
| 11 | F | 1945 | Oct-10 | 0 | 0 | 7.5 | 0 |
| | | | Apr-11 | 0 | 0 | 7.5 | 0 |
| | | | Oct-11 | 0 | 0 | 7.5 | 0 |
| | | | Apr-12 | 20 | 5 | 8 | 0 |
| | | | Oct-12 | 20 | 5 | 0.5 | 0 |
| 12 | F | 1945 | May-11 | 10 | 2 | 5.5 | 12 |
| | | | Dec-11 | 10 | 2 | 11 | 13.5 |
| | | | Jul-12 | 22.5 | 5.5 | 0 | 11.5 |
| 13 | M | 1931 | Apr-10 | 0 | 0 | 8.5 | 0.5 |
| | | | May-11 | 0 | 0 | 9.5 | 0 |
| | | | Nov-11 | 0 | 0 | 9 | 0 |
| | | | May-12 | 18 | 0 | 0 | 0 |
| 14 | M | 1946 | May-10 | 0 | 0 | 0.5 | 0 |
| | | | May-12 | 0 | 0 | 12 | 0 |
| | | | Nov-12 | 0 | 0 | 12 | 0 |
| 15 | F | 1934 | Mar-09 | 0 | 0 | 8.5 | 9 |
| | | | Apr-10 | 10 | 0.5 | 10 | 11 |
| | | | May-11 | 7.5 | 0.37 | 9.5 | 8.5 |
| | | | May-12 | 5 | 1 | 9.5 | 9 |
| | | | Dec-12 | 60 | 15 | 9.5 | 9.5 |
| 16 | F | 1949 | Apr-12 | 0 | 0 | 0 | 13.5 |
| | | | Oct-12 | 20 | 5 | 11.5 | 14.5 |
| | | | Nov-12 | 20 | 5 | 10 | 9.5 |
| 17 | M | 1941 | Mar-10 | 0 | 0 | 7.5 | 0.5 |
| | | | Sep-10 | 0 | 0 | 6.5 | 0.5 |
| | | | Apr-11 | 0 | 0 | 0 | 0.5 |
| | | | Sep-11 | 0 | 0 | 0.5 | 0 |
| 18 | M | 1943 | Feb-09 | 0 | 0 | 6.5 | 9.5 |
| | | | Jul-10 | 0 | 0 | 0.5 | 10.5 |
| | | | Jan-11 | 10 | 2 | 0.5 | 9.5 |
| | | | Jul-11 | 10 | 2 | 0 | 8.5 |
| | | | Jan-12 | 10 | 2 | 0.5 | 8 |
| | | | Jun-12 | 20 | 5 | 0.5 | 8 |
| | | | Dec-12 | 20 | 5 | 0.5 | 4 |
| 19 | M | 1941 | Sep-08 | 0 | 0 | 0 | 0.5 |
| | | | Sep-09 | 0 | 0 | 0.5 | 0 |
| | | | Jan-10 | 0 | 0 | 0.5 | 0.5 |
| | | | Sep-10 | 0 | 0 | 0 | 7.5 |
| | | | Oct-11 | 10 | 2 | 0 | 8.5 |
| | | | Apr-12 | 20 | 5 | 0 | 0.5 |
| | | | Oct-12 | 20 | 5 | 0 | 0 |
| 20 | F | 1944 | Jun-09 | 10 | 0.5 | 0.5 | 0 |
| | | | Dec-09 | 10 | 0.5 | 0 | 0 |
| | | | Jun-10 | 5 | 0.25 | 10.5 | 0 |
| | | | Dec-10 | 5 | 0.25 | 8 | 0 |
| | | | Jul-12 | 5 | 0.25 | 2.0 | 0 |
| | | | Dec-12 | 5 | 0.25 | 1.5 | 0 |
| 21 | F | 1948 | Jun-09 | 0 | 0 | 0 | 0 |
| | | | Jun-11 | 0 | 0 | 0 | 11 |
| | | | Feb-12 | 20 | 0.8 | 0.5 | 8.5 |
| | | | Sep-12 | 20 | 0.8 | 0.5 | 7.5 |
| 22 | F | 1926 | Jan-09 | 0 | 0 | 0 | 0 |
| | | | Sep-09 | 2.5 | 0.125 | 4 | 0 |
| | | | Feb-10 | — | — | 4 | 0 |
| | | | Apr-11 | 2.5 | 0.5 | 4 | 0 |
| 23 | F | 1946 | Mar-10 | 0 | 0 | 0.5 | 0 |
| | | | Oct-10 | 0 | 0 | 8 | 0 |
| | | | Apr-11 | 0 | 0 | 7 | 0 |
| | | | Nov-11 | 20 | 5 | 6.5 | 0 |
| | | | Apr-12 | 20 | 5 | 0.5 | 0 |
| 24 | F | 1954 | Jun-11 | 0 | 0 | 11.5 | 0 |
| | | | Dec-11 | 20 | 5 | 8.5 | 0 |
| | | | Jul-12 | 20 | 5 | 7 | 0 |
| 25 | M | 1949 | Jul-09 | 0 | 0 | 0 | 0 |
| | | | Mar-12 | 0 | 0 | 12 | 0 |
| 26 | M | 1929 | Apr-10 | 0 | 0 | 4.5 | 0 |
| | | | Apr-11 | 0 | 0 | 4 | 0 |
| | | | Oct-11 | 10 | 2.5 | 0 | 0 |
| | | | Apr-12 | 10 | 2.5 | 0 | 0 |
| 27 | F | 1951 | Dec-08 | 0 | 0 | 0.5 | 0.5 |
| | | | Jan-10 | 0 | 1 | 0.5 | 0.5 |
| | | | Jul-10 | 5 | 1 | 0.5 | 0.5 |
| | | | Mar-11 | 5 | 1 | 2 | 2 |
| | | | Dec-11 | 5 | 0.8 | 7.5 | 6.5 |
| | | | Jul-12 | 20 | 0.8 | 4.5 | 8 |
| 28 | M | 1941 | Jun-10 | 0 | 0 | 1 | 0.5 |
| | | | Nov-11 | 0 | 0 | 10.5 | 8.5 |
| | | | May-12 | 20 | 0.8 | 9.5 | 8.5 |
| | | | Dec-12 | 20 | 5 | 5 | 4.5 |
| 29 | M | 1930 | Oct-07 | 0 | 0 | 0 | 0 |
| | | | Jul-09 | 0 | 0 | 0 | 0 |
| | | | Jun-11 | 0 | 0 | Moderate | 0 |
| | | | | | | pucker; VA | |
| | | | | | | 20/25 | |
| | | | Dec-11 | 0 | 0 | Mild | 0 |
| | | | | | | pucker; VA | |
| | | | | | | 20/25 | |
| | | | Jun-12 | 20 | 5 | Mild | 0 |
| | | | | | | pucker; VA | |
| | | | | | | 20/25 | |
| | | | Dec-12 | 20 | 5 | Mild | 0 |
| | | | | | | pucker; VA | |
| | | | | | | 20/25 | |
| 30 | M | 1948 | Jul-09 | 0 | 0 | 9 | N/A |
| | | | Oct-09 | 5 | 0.25 | 10 | N/A |
| | | | Oct-09 | 5 | 0.25 | 10.5 | N/A |
| | | | Dec-09 | 10 | 0.5 | 10.5 | N/A |
| | | | Apr-12 | 5 | 0.25 | 11.5; plh | N/A |
| | | | | | | 0.4 cm | |
| | | | Aug-12 | 20 | 5 | 10.5; plh | N/A |
| | | | | | | 0.2 cm | |
| 31 | M | 1951 | Aug-09 | 0 | 0 | 0 | 5.5 |
| | | | Sep-10 | 0 | 0 | 0 | 5.5 |
| | | | Aug-12 | 5 | 0 | 0 | 0.5 |
| 32 | M | 1951 | Feb-10 | 0 | 0 | 0 | 0 |
| | | | Sep-11 | 0 | 0 | 11 | 0 |
| | | | Sep-12 | 20 | 5 | 5.5 | 0 |
| 33 | F | 1946 | Sep-10 | 0 | 0 | 0 | 0.5 |
| | | | Sep-11 | 0 | 0 | 0 | 0.5 |
| | | | Mar-12 | 20 | 5 | 0 | 0.5; |
| | | | | | | | Cataract |
| | | | Sep-12 | 5.7 | 1.4 | 0 | 0.5; |
| | | | | | | | Cataract |
| 34 | F | 1937 | Jan-09 | 0 | 0 | 0.5 | 12 |
| | | | Jan-10 | 0 | 0 | 0 | 13 |
| | | | Nov-10 | 0 | 0 | 0 | 15.5 |
| | | | Jun-11 | 0 | 0 | 0 | 15 |
| | | | Feb-12 | 10 | 2 | 0 | 6.4 |
| | | | Aug-12 | 10 | 2 | 0 | 3.8 |
| 35 | M | 1930 | Jun-07 | 0 | 0 | 0.5 | 0 |
| | | | Nov-10 | 0 | 0 | 12 | 0 |
| | | | Jun-11 | 10 | 0 | 9.5 | 0 |
| | | | Aug-12 | 10 | 0 | 4.5 | 0 |
| 36 | F | 1952 | Oct-10 | 0 | 0 | 11.5 | 9 |
| | | | Aug-12 | 5 | 1 | 11 | 6 |
| 37 | M | 1946 | Jun-09 | 0 | 0 | 0 | 0 |
| | | | Jun-10 | 0 | 0 | 0 | 0.5 |
| | | | Jun-11 | 0 | 0 | 0.5 | 1 |
| | | | Jul-12 | 0 | 0 | 0 | 9 |
| 38 | F | 1946 | Jan-10 | 0 | 0 | 0 | 4.3 |
| | | | Jan-12 | 0 | 0 | 0 | 8 |
| | | | Jul-12 | 20 | 5 | 0 | 0 |
| 39 | M | 1942 | Nov-09 | 0 | 0 | 0 | 0 |
| | | | May-10 | 10 | 0.5 | 0 | 0 |
| | | | Aug-10 | 10 | 0.5 | 10.5 | 2.5 |
| | | | Nov-10 | 10 | 0.5 | 10.5 | 2.5 |
| | | | Apr-12 | 10 | 0.5 | 9.5 | 2.5 |
| 40 | F | 1957 | Jan-10 | 0 | 0 | 6.4 | 0 |
| | | | Jul-10 | 10 | 2 | 5.1 | 0 |
| | | | Jan-11 | 10 | 2 | 4.6 | 0 |
| | | | Jul-11 | 10 | 2 | 4.3 | 0 |
| | | | Feb-12 | 10 | 2 | 3.2 | 0 |
| | | | Mar-12 | 10 | 2 | 2.7 | 0 |
| 41 | M | 1970 | Sep-12 | 0 | 0 | — | — |
| | | | Oct. 11, 2012 | 10 | 2 | — | — |
| | | | Oct. 18, 2012 | 0 | 0 | 4 | 6 |
| | | | Nov. 1, 2012 | 20 | 5 | — | — |
| | | | Nov. 30, 2012 | 20 | 5 | 4 | 0 |

Results:

Treatment of retinal lesions with some combination of lutein and zeaxanthin was useful for ameliorating and decreasing the size of retinal lesions in subjects having VMT. Subjects with a total of 42 retinal lesions were treated with some combination of lutein and zeaxanthin. 59.5% of retinal lesions decreased in size following treatment, 28.5% of retinal lesions increased in size following treatment and 12% of lesions showed no change in size following treatment with some combination of lutein and zeaxanthin (Table 1).

Daily oral administration to a subject of 10-20 mg lutein and 0.5-5 mg zeaxanthin was sufficient to decrease retinal lesions in the majority of subjects who had not previously been treated with Lutein and/or zeaxanthin (Table 2).

TABLE 2
Change in retinal lesion size in patients treated with a
combination of 10-20 mg lutein and 0.5-5 mg zeaxanthin.
N/A is provided where no lesion was present. Negative
values indicate lesions that decreased in size following
treatment. Positive values indicate lesions that increased
in size following treatment.

| | Change in OD | Change in OS |
|---|---|---|
| Case Number | Lesion Size (units) | Lesion Size (units) |
| 6 | −3.0 | 0.0 |
| 11 | −7.0 | N/A |
| 16 | +10.0 | −4.0 |
| 18 | 0.0 | −6.5 |
| 19 | N/A | −7.5 |
| 21 | +0.5 | −3.5 |
| 23 | −6.5 | N/A |
| 24 | −4.5 | N/A |
| 26 | −4.0 | N/A |
| 28 | −5.5 | −4.0 |
| 32 | −5.5 | N/A |
| 34 | N/A | −11.2 |
| 38 | N/A | −8.0 |
| 39 | +9.5 | +2.5 |
| 40 | −3.7 | N/A |

Twenty-one lesions were treated in 15 patients. Twelve OD lesions treated, eight decreased with treatment, three increased, one showed no change. Nine OS lesions were treated, seven decreased in response to treatment, one increased and one neither increased nor decreased. In total, 21 lesions were treated, 71% decreased in response to treatment, 19% increased in response to treatment and 10% showed no change.

Daily administration to subjects of 20 mg lutein and 5 mg zeaxanthin was sufficient to decrease lesions in the majority of subjects who had not previously been treated with Lutein and/or zeaxanthin (Table 3).

TABLE 3
Change in retinal lesion size in patients treated with a
combination of 20 mg lutein and 5 mg zeaxanthin. N/A is
provided where no lesion was present. Negative values
indicate lesions that decreased in size following treatment.
Positive values indicate lesions that increased in size
following treatment.

| | Change in OD | Change in OS |
|---|---|---|
| Case Number | Lesion Size (units) | Lesion Size (units) |
| 6 | −3.0 | 0.0 |
| 11 | −7.0 | N/A |
| 16 | +10.0 | −4.0 |
| 23 | −6.5 | N/A |
| 24 | −4.5 | N/A |
| 32 | −5.5 | N/A |
| 38 | N/A | −8.0 |

Nine lesions treated in seven patients. Seven lesions decreased in response to treatment (78%), one increased in response to treatment (11%) and one lesion remained the same size (11%).

Discussion:

Observational data suggests that increasing the daily dosage of lutein to about 20 mg and increasing the daily dosage of zeaxanthin to about 5 mg results in efficient decrease of retinal lesion size in subjects with VMT (Table 1, in particular cases 1, 2, 6, 11, 12, 19, 23, 24, 28, 32 and 38).

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

1. A method for treating a retinal disorder, the retinal disorder being vitreomacular traction disorder, the method comprising administering to a subject in need thereof a therapeutically effective amount of a composition comprising lutein and zeaxanthin.

2. (canceled)

3. The method of claim 1, wherein the retinal disorder is a nutritional deficiency disorder.

4. The method of claim 1, wherein the therapeutically effective amount of lutein is about 10 to 20 mg daily.

5. The method of claim 1, wherein the therapeutically effective amount of zeaxanthin is about 0.5-5 mg daily.

6. The method of claim 1, wherein 20 mg lutein and 5 mg zeaxanthin are administered to the subject daily.

7. The method of claim 1, wherein the composition is administered orally.

8. The method of claim 1, wherein the composition is administered daily for two to 24 months.

9. The method of claim 1, wherein the composition further comprises at least one of omega 3 fatty acids and probiotics.

10. The method of claim 9, wherein the fatty acids are at least one of Docosahexaenoic acid (DHA) and Eicosapentaenoic acid (EPA).

11. The method of claim 9, wherein the probiotics are live microorganisms belonging to the order Lactobacilliales or the genus Bifidobacterium.

12-20. (canceled)

21. The method of claim 1, wherein the composition further comprises Eicosapentaenoic acid (EPA).

22. The method of claim 21, further comprising probiotics.

23. The method of claim 22, wherein the probiotics are live microorganisms belonging to the order Lactobacilliales or the genus Bifidobacterium

24. The method of claim 21, wherein the composition further comprises Docosahexaenoic acid (DHA).

25. The method of claim 22, wherein the composition further comprises Docosahexaenoic acid (DHA).

26. The method of claim 23, wherein the composition further comprises Docosahexaenoic acid (DHA).
